(19) United States
(12) Patent Application Publication
(10) Pub. No.: US 2016/0143541 A1
(43) Pub. Date: May 26, 2016

(54) System and Method For Acousto-Electromagnetic Neuroimaging

(71) Applicants: Bin He, Arden Hills, MN (US); Kai Yu, Falcon Heights, MN (US)

(72) Inventors: Bin He, Arden Hills, MN (US); Kai Yu, Falcon Heights, MN (US)

(21) Appl. No.: 14/947,326

(22) Filed: Nov. 20, 2015

Related U.S. Application Data
(60) Provisional application No. 62/082,380, filed on Nov. 20, 2014.

(51) Int. Cl. A61B 5/00; A61B 5/04; A61B 5/0476

(52) U.S. Cl. CPC A61B 5/0093; A61B 5/0476; A61B 5/0035; A61B 5/04008; A61B 5/7228

(57) ABSTRACT

A system and method for determining an electrical activity in a subject using ultrasound and electromagnetics are provided. In some aspects, the method includes directing ultrasound energy to a portion of a subject's anatomy using an ultrasound system, wherein the ultrasound energy inducing a perturbation to locations in the subject's anatomy, and sensing, using sensors arranged about the subject, a plurality of electromagnetic signals representing an electrical activity of the subject. The method also includes identifying electromagnetic signals that are modulated by the perturbation, and generating a report of the electrical activity using the identified electromagnetic signals.

### CROSS-REFERENCE

This application is based on, claims priority to, and incorporates herein by reference in its entirety U.S. Provisional Application Ser. No. 62/082,380, filed Nov. 20, 2014, and entitled “SYSTEM AND METHOD FOR ACOUSTO-ELECTROMAGNETIC NEUROGIMAGING.”

### STATEMENT REGARDING FEDERALLY SPONSORED RESEARCH

This invention was made with government support under CBET-1450956 awarded by the National Science Foundation. The government has certain rights in the invention.

### BACKGROUND

The present disclosure is related to medical sensing and imaging. More particularly, the disclosure is directed to neural activity mapping using ultrasound and electromagnetics.

The past decade has witnessed an explosive growth in our ability to observe and measure brain activity in animals and humans. The ability to “understand the brain” has been the key to progress in neuroscience, to promote and protect brain health, and to develop treatments for restoring, regenerating, and repairing diseased and/or deteriorated brain functions.

Though vast amounts of data have been generated at molecular or cellular level using optical or electrophysiological techniques, or at the whole brain level using techniques such as functional magnetic resonance imaging (fMRI), there has been only limited progress in developing noninvasive human neuroimaging technologies allowing mapping dynamic brain activation with high resolution and precision. The local field potentials (LFP) from a smaller area or multi-unit activities (MUA) can also be recorded, hence giving a high temporal resolution with spatially specific recording and limited coverage. This limits the ability of studying spatially extended networks. At the macro-scale, the latest magnetic resonance imaging (MRI) techniques have produced fMRI and diffusion tensor imaging (DTI) datasets with unparalleled spatial resolution for noninvasive human brain imaging, yet these whole brain imaging techniques are limited in its temporal resolution, not providing detailed spatio-temporal information about neural circuits' functions and dynamics, as well as dysfunctions. What is currently lacking is a neuroimaging technology that can noninvasively map neuro-dynamics in human brains with high spatial and high temporal resolution.

The development of methods capable of building an integrated picture of the multi-scale functional brain networks will have a marked impact on our understanding of the healthy, diseased, and aged brain. Functional mapping techniques can be used to discern both the origin, as well as the direction, of information flow within the brain and can be used to analyze the complex pattern of interconnected neuronal networks. Characterization of these complex neural circuits and networks will enable a deeper understanding of the mechanisms by which the brain operates. It will lead to improved diagnosis for neuropathologies, such as stroke and epilepsy, better surgical planning, and the development and improvement of neural prostheses in cases of injury or disability. Such advancements could also lead to better management of pain as well as other brain disorders, such as schizophrenia, Alzheimer's disease, and depression.

Imaging brain activity is of utmost importance to understand the brain. Functional imaging modalities have been developed to understand the brain's mechanisms of action, including fMRI, electroencephalography (EEG), magnetoencephalography (MEG), functional near-infrared spectroscopy (fNIRS) and positron emission tomography (PET), etc. While these imaging modalities are noninvasive in nature and have been used widely to study human brain functions and dysfunctions, they are limited in either spatial resolution (such as EEG or MEG) or temporal resolution (such as fMRI and PET). fNIRS has the ability to measure both oxyhemoglobin and deoxyhemoglobin, yet it does not offer whole-brain coverage and has limited spatial and temporal resolution. EEG/MEG offers high temporal resolutions capturing brain dynamics, yet has limited spatial resolution to image brain activity due to the head volume conduction effect. fMRI is widely utilized for neuroscience research. However, the present resolution of 3 Tesla (T) fMRI typically used for cognitive neuroscience studies and clinical applications is few millimeter (mm) spatially (voxel size) and in the order of seconds temporally. With respect to temporal resolution, recent advances in accelerated fMRI have enabled volume-sampling rates as high as 10 Hz (although with compromised spatial resolution and signal-to-noise ratio (SNR) not capable of exploring high-frequency spontaneous brain activity). Yet, such temporal resolution is far below the neural activation in which action potentials are firing, in the order of one millisecond, or high frequency oscillations (recorded from intracranial electrophysiological recordings), which play a crucial role in normal and pathological processes.

In addition to mapping where neural circuits are firing, it is of equal importance to determine the functional role of different brain areas, i.e. functional segregation, which is essential to our understanding of mechanisms of neural circuits. To this goal, both high spatial and high temporal resolution are critical to help delineate the dynamics of different brain regions and networks interacting with each other, i.e. functional integration. It is highly desirable to have neuroimaging tools capable of mapping functional dynamics and interplay of neural circuits from noninvasive measurements.

Eelectrocorticography (ECoG) is a means of monitoring and mapping brain activity in selected patients undergoing surgical planning by implanting electrodes over the cortical surface. It offers direct capability of measuring brain electric activity in the vicinity of such activities and is well used in clinical applications, including aiding pre-surgical planning in epilepsy patients. Recent advancement in micro-ECoG (μECoG) has demonstrated the ability to map brain activity at a very fine spatiotemporal scale over broad areas in animal models [1]. μECoG suggests the means of mapping fine cortical dynamics which may potentially be brought to use in humans, expanding our ability to understand brain network dynamics at high spatial and temporal resolution. However, μECoG, like the ECoG approach, requires craniotomy with uncertain length of implantation for potential human use, which is currently not established for long-term use.

Current electric and electro-magnetic non-invasive neuromodulatory approaches like transcranial magnetic stimulation (TMS) and transcranial direct current stimulation (tDCS) have proven efficacious for inducing transient plastic changes in the human cortex. However, these technologies have poor spatial resolution, suffer from a depth focality tradeoff and experience significant attenuation at depth, making them inappropriate to effectively stimulate specific neural circuits. Transcranial focused ultrasound (tFUS) is a new and promising non-surgical low-energy technique for inducing transient plasticity with high spatial resolution, adjustable focus and low tissue attenuation. The putative mechanisms of the effect of mechanical energy introduced by ultrasound on nervous tissue is currently theoretical [2-4]. Despite this, there is mounting evidence in multiple preparations that ultrasound has a robust effect upon neural tissue. Ultrasound can noninvasively stimulate the hippocampus and motor cortex of intact mice [5, 6], modulate monosynaptic and polysynaptic spinal reflexes in cats [7] and disrupt seizure activity in cats [8], rats [9] and mice [10]. In addition, tFUS has been used safely and effectively for intact neural stimulation in mouse [5], rabbit [11] and monkey [12] and recently has been shown to also be a safe and effective method of transient transcranial cortical stimulation in humans [13, 14]. Recent work [13] demonstrated the feasibility of translating ultrasound through human cranium with minimal insertion loss and beam deformation and with high spatial resolution, validating ultrasound as an efficacious form of highly focal transient stimulation for use in humans.

Given the above, there is a continued need for noninvasively detecting and imaging brain activation and function in the brain with adequate specificity and time resolution.

### SUMMARY

The disclosure overcomes the aforementioned drawbacks by providing systems and methods for acousto-electromagnetic neuroimaging. Specifically, as described herein, focused ultrasound is integrated with electromagnetic sensing to map dynamic brain activation with high spatial and temporal resolutions. This approach can have a profound impact on cognitive neuroscience research and clinical applications, including diagnosis and treatment of a number of neurological and mental brain disorders, as well as to map the brain function in healthy population. For example, enhanced spatial and temporal resolutions with respect to detected neural activity can be used to improve management of certain patients, such as the patients suffering from epilepsy or chronic pain, and greatly promote cognitive studies directed to perception, attention and learning, and so on.

In accordance with one aspect of the disclosure, a method is provided for determining electrical activity in a subject using ultrasound and electromagnetic. The method includes directing ultrasound energy to a portion of a subject's anatomy using an ultrasound system, the ultrasound energy inducing a perturbation to locations in the subject's anatomy. The method also includes sensing, using sensors arranged about the subject, a plurality of electromagnetic signals representing an electrical activity of the subject. The method further includes identifying electromagnetic signals that are modulated by the perturbation and generating a report of the electrical activity using the identified electromagnetic signals.

In accordance with another aspect of the disclosure, a system is provided for determining neural activity in a subject using ultrasound and electromagnetic. The system includes a plurality of sensors capable of detecting electromagnetic signals associated with an electrical activity of a subject and an ultrasound system configured to direct ultrasound energy to a portion of a subject's anatomy. The system also includes a computer programmed to control the ultrasound system to induce a perturbation to locations in the subject's anatomy and receive electromagnetic signal data from sensors arranged about the subject. The computer is further programmed to identify, from the electromagnetic signal data, signals modulated by the perturbation and determine spatial information related to modulated signals. The computer is also programmed to generate a report of the electrical activity in the subject using the electromagnetic signal data and determined spatial information.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The present disclosure will hereafter be provided with reference to the accompanying drawings, wherein like reference numerals denote like elements.

FIG. 1 is a block diagram showing an example of a system for use in accordance with aspects of the present disclosure.

FIG. 2 is a schematic diagram illustrating systems and methods for acousto-electromagnetic neuroimaging illustrating in accordance with aspects of the present disclosure.

FIG. 3 is a flow chart showing some examples of steps of a process in accordance with aspects of the present disclosure.

FIG. 4 is a schematic diagram illustrating an experimental set-up for non-invasively mapping neural activity in a subject.

FIG. 5 is an illustration showing averages of ultrasound-induced electrical potentials.

FIG. 6 is a set of graphs showing effects of ultrasound intensities on the induced electrical signals over the scalp.

FIG. 7 is a graph and associated topographic voltage maps illustrating a response to ultrasound perturbation.

FIG. 8 is a graph showing 1/f MEG instrumentation noise.

FIG. 9A is an illustration of a modulated ultrasound waveform shifting the spectrum of neural activities.

FIG. 9B is an illustration of a modulated waveform that can be used in accordance with FIG. 9A.

FIG. 10 is a side cross-sectional view of an example of a wearable ultrasound modulated MEG system in accordance with the present disclosure.

FIG. 11 is a side cross-sectional view of an example of a wearable ultrasound modulated EEG system in accordance with the present disclosure.

FIG. 12 is a side cross-sectional view of an example of a wearable ultrasound modulated MEG-EEG system in accordance with the present disclosure.

### DETAILED DESCRIPTION

The present disclosure provides a system and method for mapping neural activity of a subject, which utilizes ultrasound energy to modulate regional neural activity and uses electromagnetic sensors to record neural activity. In particular, neural signals generated, modulated, or modified with ultrasound energy, can be measured using electromagnetic sensors while a subject is at rest, performing specific tasks, or receiving stimuli. As will be described, such measured signals can then be used to reconstruct neural activation patterns in the subject with high temporal and spatial resolution.

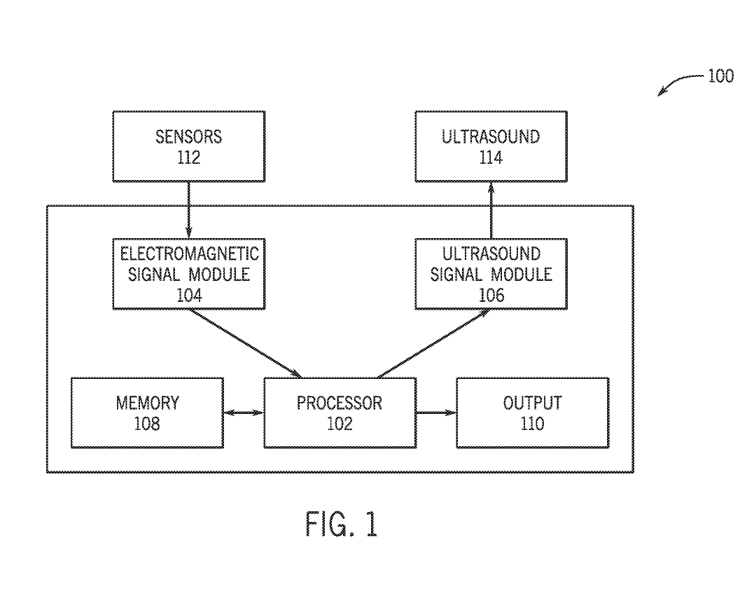

Turning to FIG. 1, an illustrative system for use in mapping neural activity in a subject, in accordance with aspects of the present disclosure, is shown. In general, the system 100 may include a processor 102 in communication with an electromagnetic signal module 104 and ultrasound signal module 106. The system 100 can also include a memory 108 and an output 110.

The processor 102 can be configured to carry out any number of actions, including storing to and retrieving data from memory 108, as well as relaying raw or processed data to output 110. In particular, the processor 102 may be configured to process electromagnetic data obtained using sensors 112 placed about a subject. In some aspects, the sensors 112 can be electrical sensors, such as wet EEG sensors or dry EEG sensors), or magnetic sensors, such as magnetic tunnel junction (“MTJ”) sensors, magnetoresistive sensors, or spintronic sensors, and so forth, or combinations thereof. By way of example, sensors 112 can be assembled in an array comprising multiple sensing elements with dimensions ranging from 100 nanometers, several tens of micrometers to a few centimeters.

Processor 102, in addition to other processing tasks, may also be configured to determine spatio-temporal neural activity by processing acquired and/or conditioned electromagnetic signal data. In addition, the processor 102 may further be configured to identify, from acquired electromagnetic signal data, signals modulated by an ultrasound 114. In addition, the processor 102 may be configured to determine spatial information related to identified modulated signals. Moreover, the processor 102 may further be configured to reconstruct neural activity from recorded signals, and generate a report indicating the neural activity and/or activation in a subject using electromagnetic signal data and determined spatial information.

The system 100 also includes an electromagnetic signal module configured to filter, amplify, condition, multiplex, and/or demodulate electromagnetic signals obtained from a subject via sensors 112. By way of example, electromagnetic signals can include electroencephalography (“EEG”) signals, magnetoencephalography (“MEG”) signals, the combination of these two signals, and the like. The system 100 also includes an ultrasound signal module 106 configured to control the ultrasound 114 to induce perturbations to various locations in the subject's anatomy by directing ultrasound energy thereabout.

Specifically, focused ultrasound (“FUS”) has been investigated for a range of neurological applications ranging from stimulation to ablative treatments. Specifically, transcranial FUS (tFUS) has been investigated as means of neurostimulation. Results have indicated effects on neural responses in humans, nonhuman primates, and small-animal models. Therefore, in some aspects, the ultrasound 114 may be configured to direct focused ultrasound energy to a subject's skull. Furthermore, a plurality of tFUS beams may be used to generate more focused areas within the brain for a better selection, such as the overlapping area of the multiple tFUS beams. However, the mechanisms by which tFUS produces the observed effects remain unclear, largely due to the lack of knowledge of the actual field distribution inside the brain. The challenges stem from the large attenuation and wave distortion through the skull, which could result in reduced focusing gain and/or spatial shift at a focal spot. Hence, the application of ultrasound arrays with transcranial refocusing capabilities may be used to address the problem of the acoustic field distortion.

The perturbation generated using the ultrasound 114 may be implemented by using a broad range of ultrasound operational parameters to optimize the spatial resolution, including using various operating frequencies, transducer apertures, operating bandwidths, coded excitations and so forth. In addition, a range of temporal modulation patterns can be utilized to produce tissue property changes with spatial and temporal profiles detectable by specific electromagnetic sensing approach utilized. In some aspects, frequencies may be in a range that avoid 1/f noise in sensed signals, such as 1 kHz to 1000 kHz, although other values may be possible.

In addition, the system 100 may further include an output 110, which may be take any shape or form, as required or desired, including a display configured to provide a clinician or researcher information regarding determined neural activity. In some aspects, the report may include, for example, two or three-dimensional maps, or images, of the neural activity of a subject. The report may further include dynamic two or three-dimensional maps or images to represent spatio-temporal distributions of neural activity.

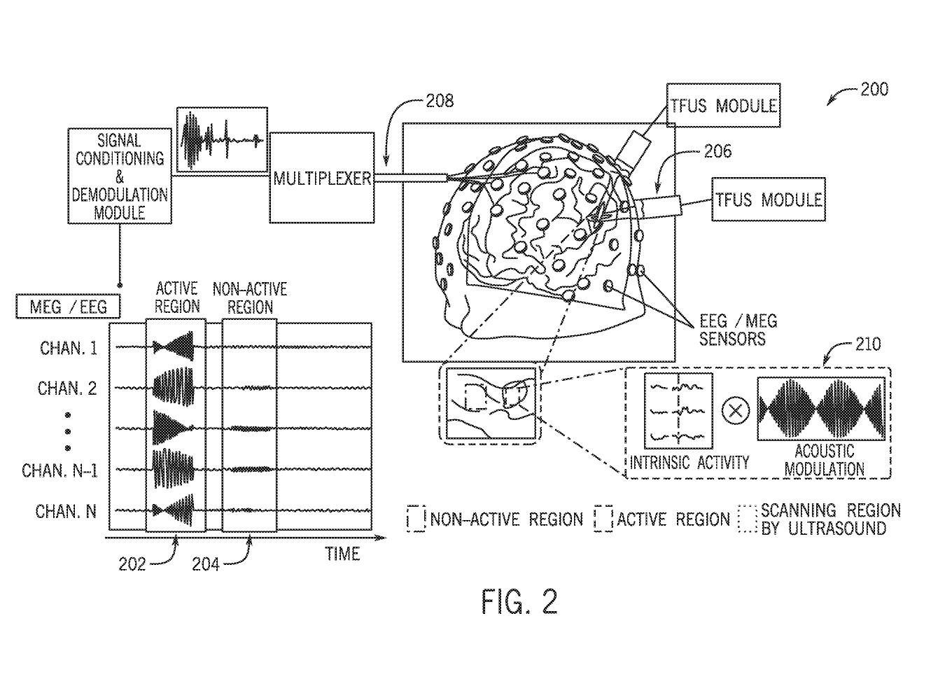

Turning to FIG. 2, an illustrative diagram of an acousto-electromagnetic neuroimaging approach, in accordance with aspects of the present disclosure, is shown. In particular, for a given brain, brain activation can consist of active regions where neural activation is encoded, and non-active regions where no synchronized neural activation may be observed. In some modes of operations, a focused ultrasound beam can be used to scan through various brain regions resulting in electrical recordings of active regions 202 and non-active region 204. Use of multiple (2 as shown in the example of FIG. 2) focused ultrasound beams can select a highly localized region. Similarly, MEG sensors can be used to record magnetoencephalographic signals of neural activity as modulated by focused ultrasound. And a combination of EEG sensors and MEG sensors can be used to record electromagnetic signals of neural activity as modulated by focused ultrasound.

Ultrasound pulses sent by a focused transducer 206 will cause a mechanical vibration of neural tissues in the targeted region, thus leading to ultrasound modulated electric/magnetic signals 210 to be detected over the scalp. Ultrasound energy will also cause a change in the local electrical properties due to the acoustoelectric effect when traveling in the brain. The electric/magnetic field perturbation due to the mechanical movement of tissue and local resistivity change may then be measured by multiple sensors 208 set on the surface of the scalp. By scanning multiple regions in the brain using focused ultrasound, the selected source area can generate detectable electric potential (or magnetic) field that can be decoded to extract information that can be used for source imaging. Such source imaging can be performed by setting up a head-brain forward model and source model, and estimating source distributions by minimizing the difference between measured ultrasound-mediated electric/magnetic signals with model predicted such signals, with known brain regions being modulated by the focused ultrasound beam(s). By combining ultrasound focusing and scanning with electromagnetic measurements, in accordance with the present disclosure, a high spatial resolution brain activation can be noninvasively estimated with a good temporal resolution, thus fitting itself in real-time brain mapping.

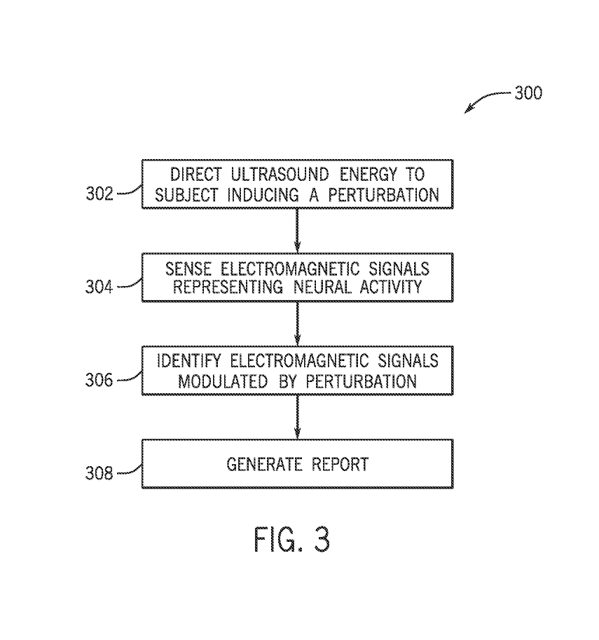

Turning to FIG. 3, a flowchart setting forth steps of a process 300 for mapping neural activity of a subject, in accordance with aspects of the present disclosure, is shown. The process may begin at process block 302 whereby ultrasonic energy, such as focused ultrasound energy, is directed to various locations within a subject's anatomy, whereby the subject may be at rest, performing specific tasks, or receiving stimuli. For example, a subject may be provided with a stimulation pattern, such as a stimulation pattern that includes binocular high-contrast square-wave moving and rotating gratings.

In some aspects, various ultrasound frequencies and modulation patterns may be utilized. At the same time, each focal location can be temporally modulated differently in a manner that may allow for identifying responses after detection with an appropriate electromagnetic sensing system. This approach would be beneficial in practicing faster imaging by sensing the induced signals from multiple locations simultaneously. In some aspects, external conditions may also be modified at process block 302. For example, a static magnetic field (B₀) may be introduced to enhance the electroacoustic effect.

At process block 304, electromagnetic signals representative of neural activity and/or neural activation may be sensed using sensors arranged about the subject. For instance, electric or magnetic signals may be recorded using an array of sensors placed at various locations about or contacting the scalp of the subject. At process block 306 electromagnetic signals modulated by the ultrasound energy may be identified, and utilized to determine spatial information related to modulated signals, to determine spatial characteristics of neural activity. In some aspects, source imaging may be performed at process block 306 using sub-space imaging algorithms or weighted minimum norm algorithms in order to reconstruct neural activation. In dependence of the nature of extracted signals, namely electric or magnetic signals, an optimization process for the source imaging may be performed. In some aspects, neural activity or activation information or imaging may be combined with or compared to information generated using other imaging modalities, such as, for example fMRI imaging. In some aspects, source imaging from electromagnetic signals recorded, after demodulation, can be performed using distributed current density models and model parameters estimated by minimizing the difference between the recorded electromagnetic signals and the model predicted signals over the sensor locations. In some aspects, such source imaging can be performed with electromagnetic signals over a plurality of time instances at a plurality of sensor locations, to realize spatio-temporal source imaging by minimizing the difference between recorded spatio-temporal distributions of electromagnetic signals and model predicted ones. In some aspects, such source imaging can be performed using discrete current dipole models to minimize the difference between recorded electromagnetic signals and model predicted ones, at a given time instant, or over a period of time consisting of a plurality of time instances. In this source imaging, head volume conductor models can be used based upon anatomical information derived from various imaging approaches, such as anatomic MRI images. These head volume conductor models can include the boundary element model [15] or finite element model [16] or other models. More generally, anatomical image information may be derived using a variety of imaging modalities, including MRI, such that anatomical images may be spatially correlated or registered with the acquired information to generate reports or maps that provide both functional and anatomical information to the user. For example, an MRI system may be used to acquire T₁-weighted MRI images from the subject, over which electrical activity can be imaged and localized from ultrasound modulated electromagnetic signals.

Then, at process block 308, a report is generated, of any shape or form. In some aspects, the report may include, for example, two or three-dimensional maps, or images, of the neural activity of a subject. The report may further include dynamic two or three-dimensional maps or images to represent spatio-temporal distributions of neural activity.

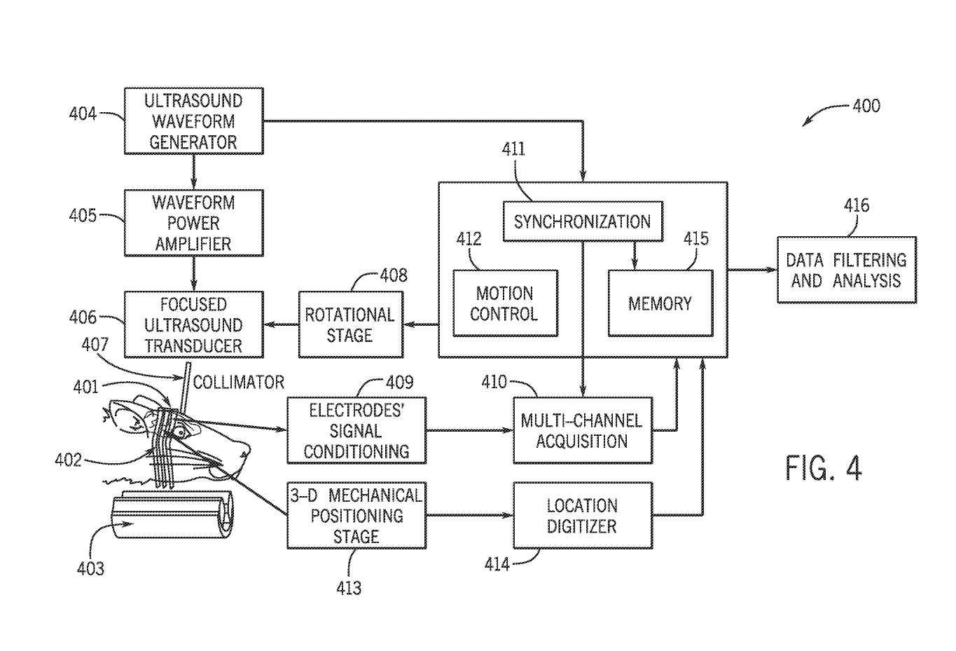

Turning to FIG. 4, an example of a system for use in an animal experiment is illustrated. In the experiment, non-invasive neural activity mapping in a rat subject was performed. However, one of ordinary skill in the art will readily appreciate the extension of this experimental system to the use with other subjects, including humans. In the preparation of such rat subjects, they are anesthetized with Katamine/Xylazine mixture with certain dosage (1 mL in average for each injection) determined by the rat's weight. Their hair over the scalp is removed to have skin exposed to EEG electrodes (or MEG sensors) 401. The electrodes are fabricated into a small volume (i.e. 2 mm×3 mm×1 mm), and are fixed to an elastic band 402. This band wraps around the rat head and a dedicated neck piece 403, such that the electrodes are mechanically forced onto the scalp while the rat can smoothly maintain its breathing. Capped with the electrodes 401, the rat head is further fixed with a mouth piece and ear bars in a small animal stereotaxic frame. For EEG electrodes, EEG paste is desirable to improve the electrical contact between the electrodes and the scalp.

In an in vivo neural imaging experiment, an ultrasound waveform generator 404 can be configured with and further produce customized sonication sequences. In particular, this ultrasound waveform may be processed with a waveform power amplifier 405, so as to drive a focused ultrasound transducer 406. A PTFE needle/funnel may be used as an ultrasound collimator 407 to collect ultrasound energy and to pass acoustic waves into a specific brain region. Ultrasound coupling gel may be used to fill the collimator 407. A rotational stage 408, fed with a motion control module 412, may be used to steer the orientation of the focused ultrasound transducer 406.

The recordings from the electrodes are pre-processed by electrodes' signal conditioning module 409, and further digitized by multi-channel acquisition module 410. This acquisition timing can be synchronized from the trigger signal produced by the ultrasound waveform generator 404 and through the synchronization module 411. A 3-dimentional mechanical positioning stage 413 is used to identify the relative locations of each electrodes over the scalp, and a location digitizer 414 is applied to record the translational movement along the x, y, and z directions. The acquired neural signals, electrodes' locations, and the sonication events are all stored into a compiled file in the memory module 415 for any further processing. In the data filtering and analysis module 416, the acquired EEG (or MEG) data are further processed by removing the electrocardiography (ECG) components using independent component analysis.

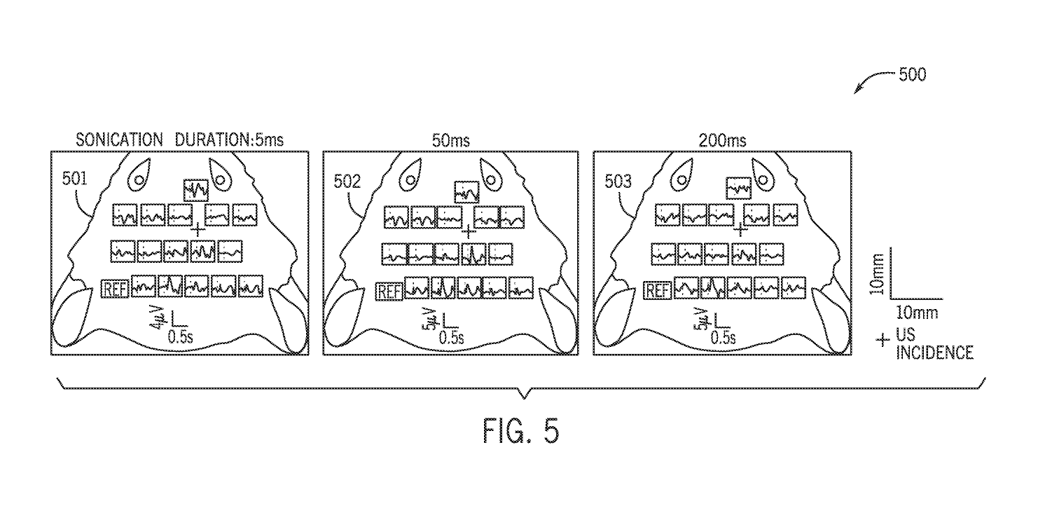

Turning to FIG. 5, an illustration is provided that shows averages of ultrasound-induced electrical potentials recorded with a 16 channel EEG in response to 5 ms, 50 ms and 200 ms sonications with medium intensities in a top-view of the rat head shown in 501, 502, and 503 respectively. FIG. 5 indicates the capability of inducing neural responses by tFUS in an in vivo animal brain.

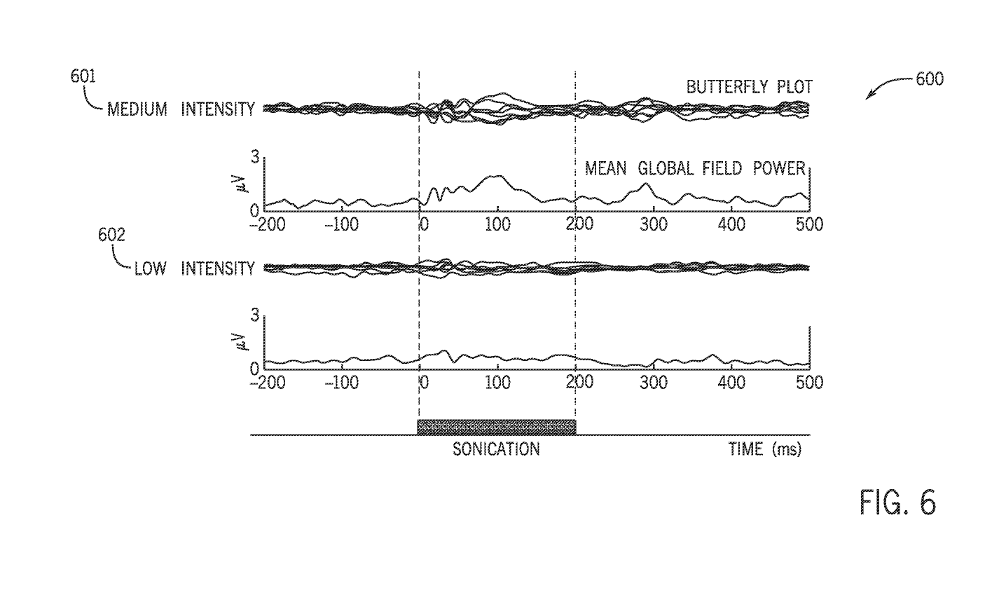

Turning to FIG. 6, when the ultrasound intensities change due to the waveform power amplifier 405 of FIG. 4, a medium intensity 60 sonication can initiate significant neural activities in response to the tFUS stimulation, illustrated with both the butterfly plot and the mean global field power profile. When the ultrasound intensity decreases to a low intensity 602 sonication, the resulting neural activation is reduced as the mean global field power does not change within the sonication period; however, a mechanical-wave modulation effect is still observed from the butterfly plot. FIG. 6 shows that the systems and methods of the present disclosure can be implemented in multiple modes. For example, when using low ultrasound intensity, no neural activation will be induced in response to tFUS, and the electromagnetic signals recorded can be used to map intrinsic neural activation in the ultrasound selected brain regions; and when using high ultrasound intensity, neural activation can be induced in response to tFUS at specific sites within the brain, thus allowing perturbation based neuroimaging mapping brain functions and networks.

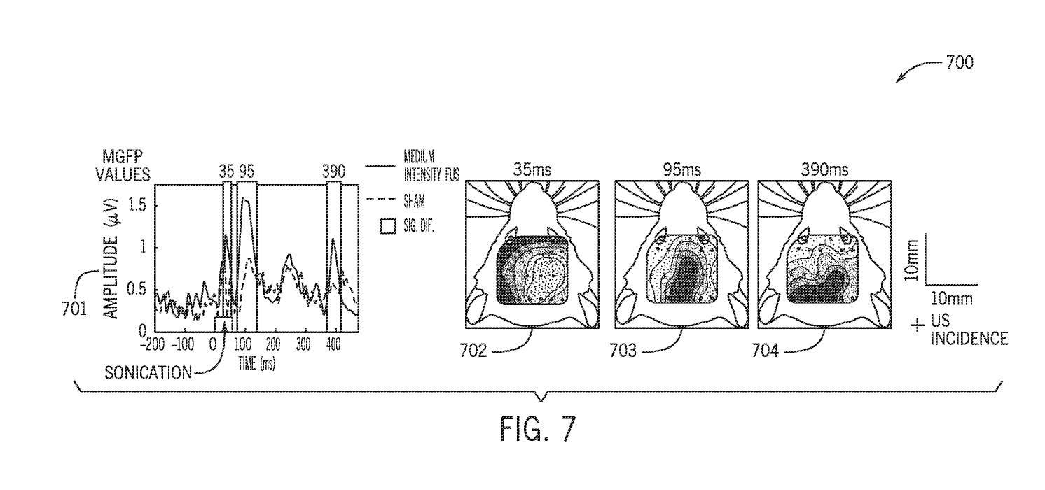

Turning to FIG. 7, for the medium intensity 601 sonication case of FIG. 6, topographic voltage maps at 35 ms, 95 ms, and 390 ms, are shown as 702, 703, and 704, respectively. These maps 702, 793, 704 correspond to the time points of interest indicated with the vertical gray bars at 35, 95, and 390 ms in 701. The white rectangular box starting at 0 ms shows period of sonication. In this case, the concept of the ultrasound-mediated neural perturbation imaging is illustrated, by mapping electrical signal distribution spatially at multiple time instances that are induced by tFUS perturbation.

In one aspect of the disclosure, dynamic electrophysiological source distributions encode the spatio-temporal pattern of brain activity. A system allowing for electrophysiological source imaging via tFUS modulation will translate to locations of the distribution of active brain regions (i.e. where a synchronous neuronal activity forming an electric/magnetic dipole vibrating exists with ultrasound wave at the time of external electromagnetic recording, and non-active regions where no coherent neuronal activity pertaining to the functional task is happening). A tFUS beam scans through brain regions where potential sources may be located at which both active and non-active regions are present. The ultrasound pulse will cause a mechanical movement of tissue in the targeted region causing ultrasound modulated electromagnetic signals to be detected over the scalp. The electromagnetic field perturbation due to the mechanical movement of tissue will then be measured by sensor arrays set on the scalp. By scanning through the brain regions using tFUS, the active area generates detectable ultrasound-modulated high frequency electromagnetic fields due to the coherent underlying neuronal activity while non-active area will produce noisy outputs due to the incoherent activities. The recorded electromagnetic signals can be decoded to extract information that reflect electrophysiological sources at active volume as selected and scanned by the tFUS beam. Using an equivalent current dipole model for a small brain volume such as 1 mm³, the location of the current dipole can be determined by the location of the center area of tFUS beam while the dipole moment can be well estimated from the scalp electrical or magnetic recordings in a least squared sense [17]. The brain current density distribution can be reconstructed using the principle of linear superposition [17] without the need of solving an ill-posed inverse problem. Due to the fact that many more sensors are available than the source parameters for a small brain volume selected by the tFUS beam (3 parameters for an equivalent dipole with fixed location), the problem becomes a well-defined over-determined estimation. With ultrasound scanning, high resolution brain activation can be noninvasively estimated. Due to the capability of fast ultrasound scanning, the present disclosure provides systems and methods for high spatio-temporal resolution for human brain mapping.

In one aspect of the disclosure, systems and methods for acousto-modulated electrical source imaging are provided. A plurality of electrode sensors can be used to record electrical signals over the scalp with tFUS modulation. Each of electrical sensors are connected through a multiplexer for further signal conditioning with an ultra-low noise, pre-amplifiers having a high input impedance, a high common mode rejection ratio, and a high sampling rate across all individual channels. 24-bit ADC (i.e. analog to digital converter) facilitates a high-resolution digitization for the acquired electric/magnetic signals, which allows to have a 20 nV/bit sampling accuracy. Different resolution ADC can be used for different applications.

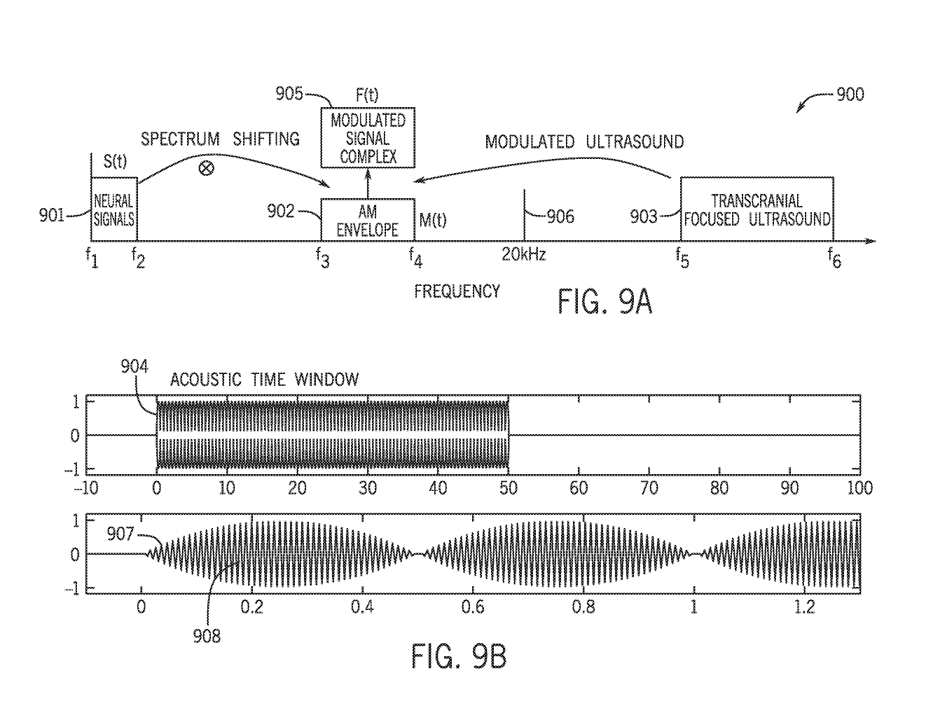

Turning to FIG. 9A, it illustrates the overall principle in accordance with the aforementioned mechanism of ultrasound-mediated, electromagnetic-detection-based neuroimaging. The neural signals 901 covers a frequency band from ƒ₁ to ƒ₂, e.g. 0.1-80 Hz, and the transcranial focused ultrasound 903 has the working frequency ranging from ƒ₅ to ƒ₆, in which the ƒ₅ is beyond the sonic frequency threshold 906 of 20 kHz, e.g. 500 kHz. Using a low-frequency modulated ultrasound, the introduced acoustic vibration can reach under the threshold 906, i.e. the amplitude modulated (AM) envelope 902 having a frequency coverage from ƒ₃ to ƒ₄, e.g. 1-10 kHz. The modulated ultrasound shifts the original spectrum of the neural signals from a low-frequency span to a relatively high-frequency zone, and thus the modulated signal complex 905 is produced and can be further processed and extracted to reconstruct the original neural signals 901. As an example in FIG. 9B, the AM envelope 902 can be a series of sinusoidal oscillations 907 with a certain acoustic time window 904, in which the focused ultrasound 908 works as a carrier wave.

In one aspect of the disclosure, the recorded electrical signals, whose segments can be featured by the known kilohertz sinusoidal sonication wave 902 (denoted as M(t)), imply the overseen brain regions on the one hand, and the searching for such known-frequency sonication wave along the time course in recorded signal profiles (i.e. the onset of the sonication, is entitled to indicate the time window 904 that is observing the corresponding neural dynamics). Such detected electric/magnetic signal complex 905 F(t)=S(t)\hat{×}M(t) further multiplies with the known M(t) 902 to produce advanced modulated signal complexes Q(t)=F(t)\hat{×}M(t). By demodulating, the intrinsic neural signals S(t) 901 at a specific location targeted by ultrasound beam can be extracted and recovered from the detected signal complex. Current source model can be further applied to represent the neural activity at the focused region. By scanning the focused ultrasound beam over the brain, distributed brain electrical activity can be estimated from the recorded electrical signals over the scalp.

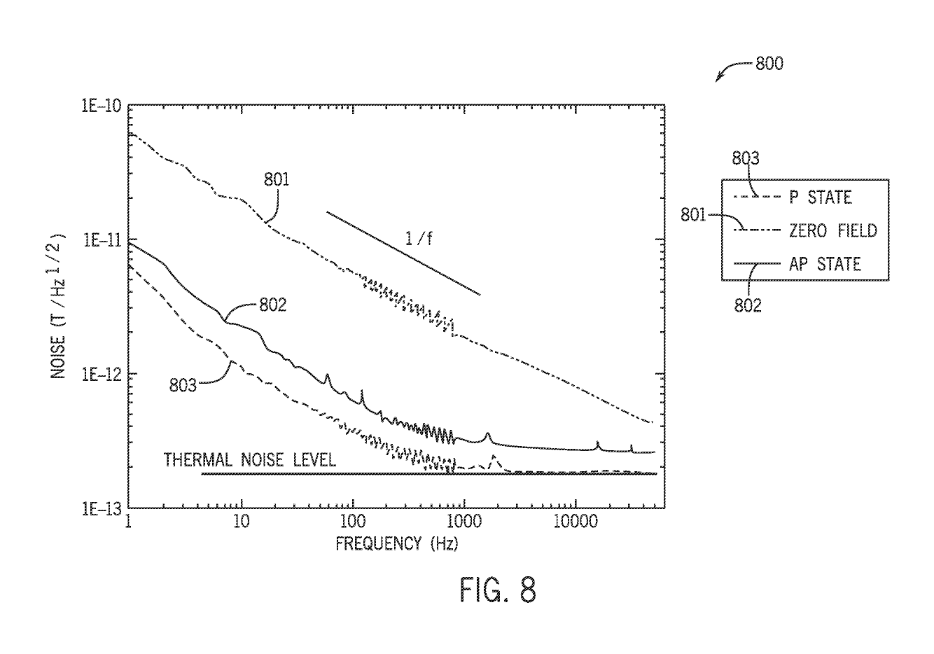

In another aspect of the disclosure, systems and methods for acousto-modulated magnetic source imaging are provided. A plurality of magnetic sensors including Spintronic sensors and tunnel magnetoresistance (TMR) sensors can be used to record magnetic signals with tFUS modulation. Each of magnetic sensors is connected to a multiplexer for signal conditioning with 24-bit ADC (analog to digital converter). Previously, reference [18] reported that a chip-scale atomic magnetometer was developed and used for an uncooled MEG acquisition. Reference [19] disclosed a rapid portable MEG device working in the room temperature. Turning to FIG. 8 which is from reference [20], the magnetic sensor noise demonstrates an inverse proportional function 801 of the frequency. Again, the sensors can include a TMR sensor design, which can detect magnetic field above where flicker noise no longer dominates. The recorded magnetic signals, having better signal-to-noise ratio are demodulated to extract neural signals at the site of ultrasound beam targeting. A current source model can be used to represent the neutral activity at the focused region. By ultrasound scanning, distributed brain electrical activity can be estimated from the scalp recorded magnetic signals.

The recorded ultrasound-modulated magnetic or electric signals are processed and envelope extracted using aforementioned procedure or Hilbert transform to demodulate the intrinsic neural signals. Time-varying instantaneous oscillation frequencies are analyzed using the frequency sliding method that allows for detailed analysis of small shifts in the peak frequency within a frequency band. The instantaneous frequency can be identified as a change in phase per unit time. This can be understood as the first temporal derivative of the unwrapped phase angle time series. The derivative is converted to hertz by multiplying by the data sampling rate in hertz and then dividing by 2π. The result is a time series of estimated instantaneous peak oscillation frequencies within the band-pass. Band passes include biological frequencies of interest including theta (4-8 Hz), alpha (6-14 Hz), beta (12-30 Hz), gamma (30-100 Hz), and high frequency oscillation (100+ Hz). With the design of tFUS scanning, the acousto-electromagnetic neuroimaging can selectively sense neural activity in focal regions selected by tFUS beams. The averaged neural activity in the selected region (e.g. 1 mm³) is sensed using electrodes or magnetic sensors over the scalp at acousto-modulated frequency. Using the equivalent dipole model representing neural activity within the selected brain region, one can uniquely reconstruct the dipole moment from scalp MEG/EEG recordings at each tFUS selected region.

In one aspect of the disclosure, room temperature MEG recording is accomplished by frequency shifting through the ultrasound modulation with frequency up to 1 kHz or up as in FIG. 9A and 9B. As used herein, room temperature may include temperatures between approximately 20 degrees C. to 26 degrees C. Ultrasound modulation allows noninvasive recording of MEG signals at high frequency above 1 kHz. This effectively and substantially reduces the typical 1/f noise per FIG. 8. Since the brain signals remain similar, the significantly-reduced instrumentation noise effectively substantially increases the signal-to-noise ratio of MEG recording, thus allowing detection of MEG signals at room temperature using various magnetic sensors such as magnetoresistance sensors, giant magnetoresistance sensors, or tunneling magnetoresistance sensors, which convert the change of a weak magnetic signal into a measurable electrical signal without consuming much electrical power. These highly sensitive magnetic sensors working without cooling system, and their miniaturized physical size (e.g., 100 nm-300 μm) can form a sensor array that enables a wearable setup for such MEG cap about the scalp, like the EEG cap on a subject. In a particular case, the MEG sensor elements can be integrated together with conventional EEG electrodes, and work jointly at room temperature.

In another aspect of the disclosure, acousto-modulated magnetic source imaging can be pursued. A plurality of magnetic sensors can be used to record magnetic signals over the scalp with tFUS modulation. Each of the magnetic sensors can be connected through a multiplexer for further signal conditioning with ultra-low noise. Pre-amplifiers having a high input impedance, a high common mode rejection ratio, and a high sampling rate can be used across all individual channels. A 24-bit analog-to-digital converter (ADC) can facilitate a high-resolution digitization for the acquired magnetic signals, which allows to have a 20 nV/bit sampling accuracy.

To this end, FIG. 9A, illustrates the overall principle in accordance with the aforementioned mechanism of ultrasound-mediated, electromagnetic-detection-based neuroimaging. The neural signals 901 covers a frequency band from ƒ₁ to ƒ₂, e.g. 0.1-80 Hz, and the transcranial focused ultrasound 903 has the working frequency ranging from ƒ₅ to ƒ₆, in which the ƒ₅ is beyond the sonic frequency threshold 906 of 20 kHz, e.g. 500 kHz. Using a low-frequency modulated ultrasound, the introduced acoustic vibration can reach under the threshold 906 (i.e. the amplitude modulated (AM) envelope 902 having a frequency coverage from ƒ₃ to ƒ₄, e.g. 1-10 kHz). The modulated ultrasound shifts the original spectrum of the neural signals from a low-frequency span to a relatively high-frequency zone, and thus the modulated signal complex 905 is produced and can be further processed and extracted to reconstruct the original neural signals 901. As an example in FIG. 9B, the AM envelope 902 can be a series of sinusoidal oscillations 907 with a certain acoustic time window 904, in which the focused ultrasound 908 works as a carrier wave. The recorded magnetic signals can have segments featured by the known kilohertz sinusoidal sonication wave 902 (denoted as M(t)), which implies the overseen brain regions on the one hand, and the searching for such known-frequency sonication wave along the time course in recorded signal profiles (i.e. the onset of the sonication, is entitled to indicate the time window 904 that is observing the corresponding neural dynamics). Such detected electrical/magnetic signal complex 905 F(t)=S(t)\hat{×}M(t) further multiplies with the known M(t) 902 to produce advanced modulated signal complexes Q(t)=F(t)\hat{×}M(t). By demodulating, the intrinsic neural signals S(t) 901 at a specific location targeted by the ultrasound beam can be extracted and recovered from the detected signal complex. A current source model can be further applied to represent the neural activity at the focused region. By scanning the focused ultrasound beam over the brain, distributed brain electrical activity can be estimated from the recorded magnetic signals over the scalp.

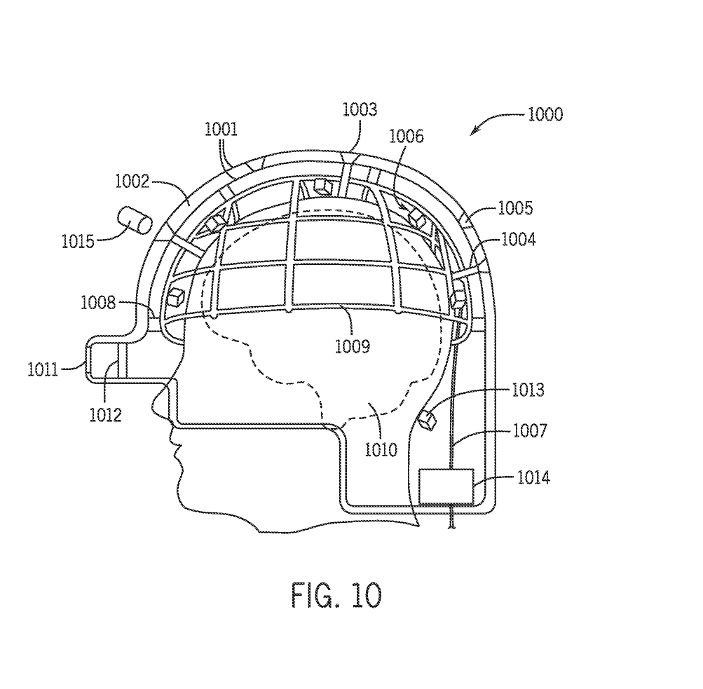

Turning to FIG. 10, a wearable ultrasound-mediated MEG device 1000 is illustrated working under the room temperature. An aluminum shielding structure 1001 is contoured to match the head of a subject. The μ-metal 1002 of the shielding structure 1001 can be designed to reduce the effect of external magnetic disturbances, and thus to improve the signal-to-noise ratio (SNR) performance of the MEG acquisition. This shielding structure 1001 and 1002 are supported by the shielding helmet supporting pillars 1008, and the head fixing net 1009, which is designed to cover the subject's head. A plurality of magnetic sensors (e.g., TMR sensors) 1006 are fixed onto the head fixing net 1009 to record the neural activity from the brain 1010. The targeted neural activity is selected by an ultrasound transducer 1015, spatially by the ultrasound focusing, and temporally by the acoustic time window 904 in FIG. 9A. The ultrasound incidence window 1003 opened through the aluminum shielding structure 1001 and the μ-metal 1002, together with an ultrasound collimator 1004, can guide the ultrasound energy input to the head coupled by ultrasound coupling gel 1005. MEG signals are read out through a MEG signal cable 1007 to deliver the signals to pre-amplifiers. A magnetic reference sensor array 1013 may be provided for noise cancellation within the acquired signals via the MEG working sensors 1006. Ambient light blinds 1011 can be provided that work with a transparent slide 1012 for device alignment. By aligning a marker on the transparent slide 1012 to an external reference, the whole ultrasound-mediated MEG device 1000 can be aligned and be further used to assist ultrasound beams aiming at a specific brain region. By turning off the ambient light blinds 1011, the brain's activity can be less affected by irrelevant stimulations/events. A carry-on battery 1014 can feed the magnetic sensor arrays 1006 and 1013 with a quality power supply.

In another aspect of the disclosure, an ultrasound-mediated MEG device can be used without the shielding structure 1001 and 1002. Magnetic sensors 1006 can be fixed onto a cap or hat to be attached onto the scalp. Magnetic sensors 1006 and 1013 can be wired or communicated via wireless network.

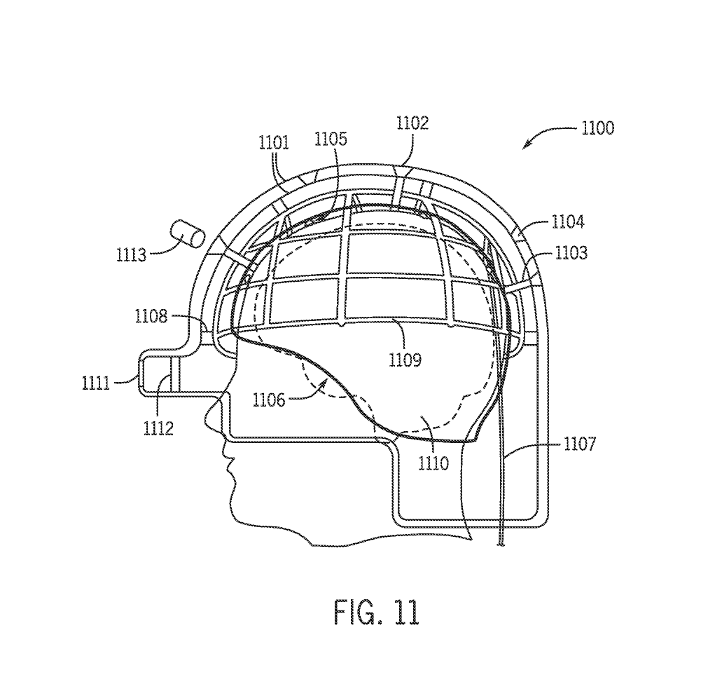

Turning to FIG. 11, a wearable ultrasound-mediated EEG device 1100 is illustrated. An aluminum shielding structure 1101 can be provided to improve the SNR performance of the acquired EEG signals. This shielding structure 1101 is supported by the shielding helmet supporting pillars 1108, and a head fixing net 1109 that is to cover a subject's head. An EEG cap 1106 underneath includes EEG electrodes 1105 (e.g. Ag/AgCl electrodes or other types of electrodes, including wet and dry electrodes) that record the neural activity from the brain 1110. The targeted neural activity is selected by the ultrasound transducers 1113, spatially by the ultrasound focusing, and temporally by the acoustic time window 904 in FIG. 9A. Both the ultrasound incidence window 1102 opened over the aluminum shielding structure 1101 and the ultrasound collimator 1103 can guide the ultrasound energy input to the scalp coupled by an ultrasound coupling gel 1104. The EEG signals are read out through an EEG signal cable 1107 to signal pre-amplifiers. Ambient light blinds 1111 can be provided to work with a transparent slide 1112 for device alignment using a visual feedback from the subject, such that the ultrasound incidence window 1102 and the ultrasound collimator 1103 can be fit into the layout of the EEG electrodes 1105. A marker, like a “cross”, may be prepared on the transparent slide 1112 and, by aligning this marker to an external reference, the whole ultrasound-mediated EEG device can be aligned and be further used to quickly assist ultrasound beams to aim at a specific brain region. This design can fix the relative positions for the ultrasound transducer 1113 and the subject's head. Previously-reported research studies required transducer tracker and head-motion tracker [22], which may increase the complexity and cost for a neuroimaging application. By turning off the ambient light blinds 1111, the brain's activity can be less affected by irrelevant stimulations/events, which can reduce artifacts in the acquired EEG.

In another aspect of the disclosure, an ultrasound-mediated EEG device can be used without the shielding structure 1101. Electrodes 1105 can be fixed onto a cap or hat to be attached onto the scalp. These sensors can be wired and connected wirelessly and communicated to processors.

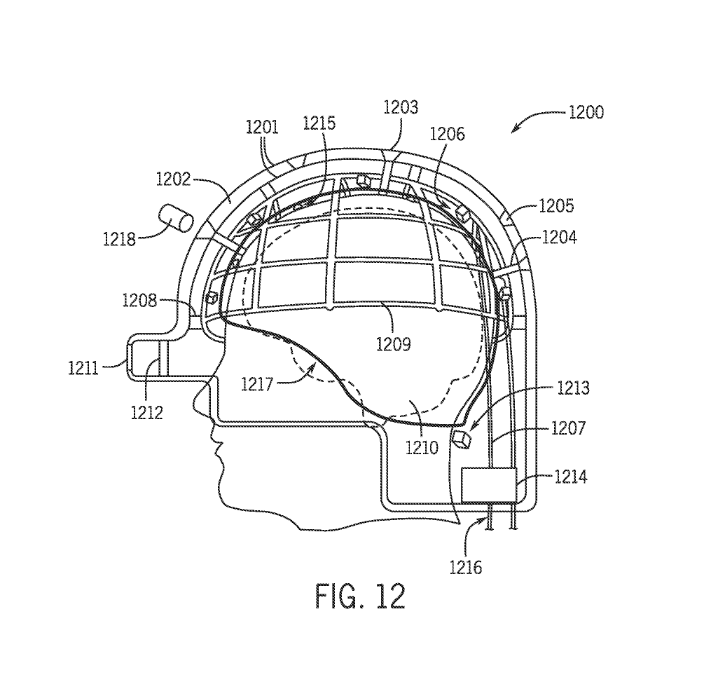

Turning to FIG. 12, a wearable acousto-electromagnetic EEG-MEG device 1200 is illustrated working under the room temperature. An aluminum shielding structure 1201 and μ-metal structure 1202 can be included to reduce the effect of external magnetic disturbances, and thus to improve the SNR performance of the EEG and the MEG acquisition. This shielding structure 1201 and 1202 is supported by shielding helmet supporting pillars 1208, and a head fixing net 1209 that covers a subject's head. A plurality of magnetic sensors, such as TMR sensors, forms a sensor arrays 1206 that is fixed onto the head fixing net 1209 to record the neural magnetic signals from the brain 1210. An EEG cap 1217 underneath includes EEG electrodes 1215 (e.g. Ag/AgCl electrodes or other types of electrodes including wet and dry electrodes) that record the neural electrical signals from the brain 1210. The targeted neural activity is selected by the ultrasound transducers 1218, spatially by the ultrasound focus, and temporally by the acoustic time window 904 in FIG. 9A. The ultrasound incidence window 1203 opened through the aluminum shielding structure 1201 and the μ-metal 1202, together with an ultrasound collimator 1204, guide the ultrasound energy input to the scalp coupled by an ultrasound coupling gel 1205. The MEG signals are read out through a MEG signal cable 1207 to signal pre-amplifiers, and the EEG signals are read out through the EEG signal cable 1216 to signal pre-amplifiers. A magnetic reference sensor array 1213 is provided to perform noise cancellation within the acquired signals via the MEG working sensors 1206. Ambient light blinds 1211 work with a transparent slide 1212 for device alignment. By aligning a marker on the transparent slide 1212 to an external reference, the whole ultrasound-mediated EEG device can be aligned and be further used to assist ultrasound beams aiming at a specific brain region. By turning off the ambient light blinds 1211, the brain's activity can be less affected by irrelevant stimulations/events. The carry-on battery 1214 can feed the magnetic sensor arrays 1206 and 1213 with a quality power supply.

Thus, the presently-described systems and methods for neuroimaging brain activity provides images with high resolutions, in both the space and time domain. The approach described herein addresses a significant technological hurdle to understanding functionality brain, and has potential significant impact to diagnosing and managing brain conditions, that cost over $500 billion each year for US alone. In addition, use of the described systems and methods may enhance quality of life for healthy population throughout the lifespan, and improve significantly the management of brain disorders, with significant impact to public health and economy.

In particular, the approach described herein utilizes ultrasound energy to provide structure identification or imaging of biological tissues in combination with electrical or magnetic sensing approaches for determining neural activity. That is, the present disclosure provides a multimodal neuroimaging technology that integrates focused ultrasound modulation with electromagnetic imaging into a single hybrid neuroimaging modality. Such capability can be used to transform the current state of the art, which achieves neuroimaging via separate modalities that can map brain activity either with high spatial or high temporal resolution.

In another aspect of the present disclosure, the acousto-electromagnetic imaging can be used to image other organ systems in a subject, such as mapping electrical activity in the heart, muscle, and the like. The principles of focused ultrasound modulation and electromagnetic sensing and imaging apply regardless of organ systems being studied.

The present disclosure has been provided in terms of one or more preferred embodiments, and it should be appreciated that many equivalents, alternatives, variations, and modifications, aside from those expressly stated, are possible and within the scope of the invention.

In one aspect of the disclosure, a neuroimaging technology by fully integrating tFUS modulation with electromagnetic source imaging into a single hybrid neuroimaging modality to accomplish the acousto-modulated electrophysiological source imaging. The tFUS is used to selectively modulate certain brain volume in a highly focused fashion to record magnetic or electric signals generated by neurons located in the focal region selected by tFUS. By scanning through the brain using tFUS beams, the magnetic/electric signals generated within the selected volume can be demodulated from the known mechanical carriers so as to decode the intrinsic neural information that can be used for electrophysiological source imaging. This approach offers the high spatial resolution of the ultrasound and the high temporal resolution of the MEG/EEG. Due to the capability of fast ultrasound scanning and demonstrated focality of tFUS, together with the high temporal resolution of electromagnetic signal conduction, the acousto-modulated electrophysiological source imaging technology promises to offer unprecedented high spatio-temporal resolution for human brain imaging.

In one aspect of the disclosure, a method for determining neural activity in a subject using ultrasound is provided. The method includes directing ultrasound energy to a portion of a subject's anatomy using an ultrasound system, wherein the ultrasound energy inducing a controlled mechanical perturbation to certain locations in the subject's anatomy and/or inducing electrical property changes according to acoustoelectric effects, and sensing, using sensors arranged about the subject, and a plurality of electromagnetic signals representing a neural activity of the subject. The method also includes identifying electromagnetic signals that are modulated by the perturbation, and generating a report of the neural activity using the identified electromagnetic signals.

In another aspect of the disclosure, a system for determining neural activity in a subject using ultrasound is provided. The system includes a plurality of sensors capable of detecting electromagnetic signals associated with a neural activity of a subject, and an ultrasound system configured direct to ultrasound energy to a portion of a subject's anatomy. The system also includes a computer programmed to control the ultrasound system to induce a perturbation to locations in the subject's anatomy, and generate modulated electromagnetic signal data from sensors arranged about the subject in corresponding to the focused modulation to the locations. The computer is also programmed to identify, from the electromagnetic signal data, signals modulated by the perturbation, and determine spatial information related to modulated signals. The computer is further programmed to generate a report of the neural activity in the subject using the electromagnetic signal data and determined spatial information.

In one aspect of the disclosure, a system for sensing and imaging neural activity in a subject using ultrasound and electromagnetics is provided. The system includes at least one focused ultrasound generator, a plurality of sensors capable of detecting electromagnetic signals associated with a neural activity of a subject being modulated by the acoustic waveforms having ultrasound frequency, and an ultrasound system configured direct to ultrasound energy to a portion of a subject's anatomy once at a plurality of locations and then to other locations in following time segments. The system also includes a decoder to extract electrophysiological signals from the ultrasound modulated electromagnetic recordings, spatially coded by the ultrasound-administered location. The system further includes a computer programmed to control the ultrasound system to induce a perturbation to locations in the subject's anatomy, and generate modulated electromagnetic signal data from sensors arranged about the subject in response to the focused modulation at the locations. The computer is also programmed to inversely localize and image current density distribution from recorded electromagnetic signals with spatially encoding of ultrasound with regard to the spatial location. The computer can be programmed to include a forward model of the volume conductor of the head and brain and neural current source models. The computer can be programmed to localize neural activity as tagged by the locations determined by one or multiple ultrasound beams. The computer is further programmed to generate a report of the neural activity in the subject using the electromagnetic signal data and determined spatial information.

In another aspect of disclosure, frequency shifting through the ultrasound modulation with frequency up to 1 kHz or up, is provided to substantially reduce the typical pink noise (i.e. 1/f noise). By stimulating a focused area, the electromagnetic signals shift the brain signals from their intrinsic low-frequency band such as up to 80 Hz to a tFUS modulating frequency of above kHz or even above 10 kHz, which allows to tremendously reduce the instrumentation noise. This aspect will allow an effective detection of brain magnetic signals with carrier of kHz level ultrasound modulating frequency. Various magnetic detection techniques such as magnetoresistance sensors, giant magnetoresistance sensors, or tunneling magnetoresistance sensor, which converts the change of a weak magnetic signal into a measurable electrical signal without consuming much electrical power, could be used to detect the magnetic signals.

In another aspect of the disclosure, tFUS can be applied to generate modulatory effects and brain responses recorded by electrical or magnetic sensors to register brain activation. In this embodiment, EEG or MEG signals can be recorded as induced by strong enough tFUS, which represent spatio-temporal distributions in response to a specific tFUS. The dynamic brain activation following such tFUS can be reconstructed from recorded EEG or MEG signals by means of source imaging. The locations and propagation of neural activation induced by tFUS can be used to probe the brain functions and dysfunctions. The tFUS-EEG/MEG approach thus provides a window to interrogate how brain works and what goes wrong in the brain networks in responding to ultrasound perturbation.

### REFERENCES

[1] J. Viventi, D. H. Kim, L. Vigeland, E. S. Frechette, J. A. Blanco, Y. S. Kim, et al., “Flexible, foldable, actively multiplexed, high-density electrode array for mapping brain activity in vivo,” Nature Neuroscience, vol. 14, pp. 1599-U138, December 2011.
[2] W. J. Tyler, “Noninvasive neuromodulation with ultrasound? A continuum mechanics hypothesis,” Neuroscientist, vol. 17, pp. 25-36, Febuary 2011.
[3] J. Mueller and W. J. Tyler, “A quantitative overview of biophysical forces impinging on neural function,” Physical Biology, vol. 11, p. 051001, 2014.
[4] M. Plaksin, S. Shoham, and E. Kimmel, “Intramembrane cavitation as a predictive bio-piezoelectric mechanism for ultrasonic brain stimulation,” Journal of Molecular Neuroscience, vol. 53, pp. S103-S103, August 2014.
[5] Y. Tufail, A. Matyushov, N. Baldwin, M. L. Tauchmann, J. Georges, A. Yoshihiro, et al., “Transcranial pulsed ultrasound stimulates intact brain circuits,” Neuron, vol. 66, pp. 681-94, Jun. 10 2010.
[6] R. L. King, J. R. Brown, W. T. Newsome, and K. B. Pauly, “Effective parameters for ultrasound-induced in vivo neurostimulation,” Ultrasound Med Biol, vol. 39, pp. 312-31, February 2013.
[7] C. N. Shealy and E. Henneman, “Reversible effects of ultrasound on spinal reflexes,” Arch Neurol, vol. 6, pp. 374-86, May 1962.
[8] J. S. Manlapaz, K. E. Astroem, H. T. Ballantine, Jr., and P. P. Lele, “Effects of Ultrasonic Radiation in Experimental Focal Epilepsy in the Cat,” Exp Neurol, vol. 10, pp. 345-56, October 1964.
[9] B. K. Min, A. Bystritsky, K. I. Jung, K. Fischer, Y. Zhang, L. S. Maeng, et al., “Focused ultrasound-mediated suppression of chemically-induced acute epileptic EEG activity,” BMC Neurosci, vol. 12, p. 23, 2011.
[10] Y. Tufail, A. Yoshihiro, S. Pati, M. M. Li, and W. J. Tyler, “Ultrasonic neuromodulation by brain stimulation with transcranial ultrasound,” Nat Protoc, vol. 6, pp. 1453-70, September 2011.
[11] S. S. Yoo, A. Bystritsky, J. H. Lee, Y. Zhang, K. Fischer, B. K. Min, et al., “Focused ultrasound modulates region-specific brain activity,” Neuroimage, vol. 56, pp. 1267-75, June 1 2011.
[12] T. Deffieux, Y. Younan, N. Wattiez, M. Tanter, P. Pouget, and J. F. Aubry, “Low-intensity focused ultrasound modulates monkey visuomotor behavior,” Curr Biol, vol. 23, pp. 2430-3, Dec. 2, 2013.
[13] W. Legon, T. F. Sato, A. Opitz, J. Mueller, A. Barbour, A. Williams, et al., “Transcranial focused ultrasound modulates the activity of primary somatosensory cortex in humans,” Nat Neurosci, vol. 17, pp. 322-9, February 2014.
[14] J. Mueller, W. Legon, A. Opitz, T. F. Sato, and W. J. Tyler, “Transcranial Focused Ultrasound Modulates Intrinsic and Evoked EEG Dynamics,” Brain Stimul, Sep. 6, 2014.
[15] M. S. Hamalainen and J. Sarvas, “Realistic conductivity geometry model of the human head for interpretation of neuromagnetic data,” IEEE Trans Biomed Eng, vol. 36, pp. 165-71, February 1989.
[16] Y. C. Zhang, S. A. Zhu, and B. He, “A second-order finite element algorithm for solving the three-dimensional EEG forward problem,” Physics in Medicine and Biology, vol. 49, pp. 2975-2987, Jul. 7, 2004.
[17] B. He, L. Yang, C. Wilke, and H. Yuan, “Electrophysiological imaging of brain activity and connectivity-challenges and opportunities,” IEEE Trans Biomed Eng, vol. 58, pp. 1918-31, July 2011.
[18] T. H. Sander, J. Preusser, R. Mhaskar, J. Kitching, L. Trahms, and S. Knappe, “Magnetoencephalography with a chip-scale atomic magnetometer,” Biomedical Optics Express, vol. 3, pp. 981-990, May 1, 2012.
[19] I. Cernak, H. W. Ko, and M. P. Mcloughlin, “Magnetoencephalography system and method for 3d localization and tracking of electrical activity in brain,” 2012.
[20] S. H. Liou and Y. Zhang, “High Sensitivity Magnetoresisitive Sensors for both DC and EMI Magnetic Field Mapping,” NEBRASKA UNIV LINCOLN, 2012.
[21] M. X. Cohen, “Fluctuations in Oscillation Frequency Control Spike Timing and Coordinate Neural Networks,” Journal of Neuroscience, vol. 34, pp. 8988-8998, Jul. 2, 2014.
[22] W. Lee, H. Kim, Y. Jung, I. U. Song, Y. A. Chung, and S. S. Yoo, “Image-guided transcranial focused ultrasound stimulates human primary somatosensory cortex,” Sci Rep, vol. 5, p. 8743, 2015.

1. A method for determining electrical activity in a subject using ultrasound and electromagnetics, the method comprising:
directing ultrasound energy to a portion of a subject's anatomy using an ultrasound system, the ultrasound energy inducing a perturbation to locations in the subject's anatomy;
sensing, using sensors arranged about the subject, a plurality of electromagnetic signals representing an electrical activity of the subject;
identifying electromagnetic signals that are modulated by the perturbation; and
generating a report of the electrical activity using the identified electromagnetic signals.

2. The method of claim 1, wherein the electrical activity is neural activity.

3. The method of claim 1, wherein the ultrasound energy is a focused ultrasound energy.

4. The method of claim 1, wherein the ultrasound energy is transcranial focused ultrasound (tFUS) and wherein identifying the electromagnetic signals that are modulated by the perturbation includes demodulating from known mechanical carriers to decode intrinsic neural information.

5. The method of claim 1, wherein the electromagnetic signals include electroencephalography (“EEG”) signals.

6. The method of claim 1, wherein the electromagnetic signals include magnetoencephalography (“MEG”) signals.

7. The method of claim 6 wherein the MEG signals are acquired at room temperature.

8. The method of claim 1, the method further comprising generating a map indicative of the electrical activity in the subject.

9. The method of claim 1 further comprising frequency shifting the ultrasound energy with frequency up to at least 1 kHz to reduce noise in the electromagnetic signals.

10. The method of claim 1 further comprising performing an anatomical imaging of the subject's anatomy and correlating the report of the electrical activity with an anatomical image of the subject's anatomy.

11. The method of claim 10 wherein the anatomical imaging includes magnetic resonance imaging (MRI).

12. The method of claim 1 wherein the electrical activity of the subject includes electrical activity from the subject's brain, muscles, heart, or organs.

13. A system for determining neural activity in a subject using ultrasound and electromagnetics, the system comprising:
a plurality of sensors capable of detecting electromagnetic signals associated with an electrical activity of a subject;
an ultrasound system configured to direct ultrasound energy to a portion of a subject's anatomy; and
a computer programmed to:
i. control the ultrasound system to induce a perturbation to locations in the subject's anatomy;
ii. receive electromagnetic signal data from sensors arranged about the subject;
iii. identify, from the electromagnetic signal data, signals modulated by the perturbation;
iv. determine spatial information related to modulated signals;
v. generate a report of the electrical activity in the subject using the electromagnetic signal data and determined spatial information.

14. The system of claim 13, wherein the electrical activity is neural activity.

15. The system of claim 13, wherein the plurality of sensors includes magnetic sensors.

16. The system of claim 15, wherein the magnetic sensors include magnetic tunnel junction (“MTJ”) sensors, magnetoresistive sensors, tunneling magnetoresistance (“TMR”) sensors, or spintronic sensors, or combinations thereof.

17. The system of claim 13, wherein the ultrasound energy is a focused ultrasound energy.

18. The system of claim 13, wherein the electromagnetic signals include electroencephalography (“EEG”) signals.

19. The system of claim 13, wherein the electromagnetic signals include magnetoencephalography (“MEG”) signals.

20. The system of claim 13, the computer further programmed to generate a map indicative of the electrical activity in the subject.

21. The system of claim 13, the computer further programmed to generate a map indicative of the neural activity in the subject.

22. The system of claim 13, wherein the ultrasounds system includes a transcranial focused ultrasound (tFUS) system and wherein the computer is programmed to identify the signals modulated by the perturbation by demodulating signals from known mechanical carriers to decode intrinsic neural information.

23. The system of claim 13, wherein the computer is further programmed to determine spatial information related to the modulated signals using electromagnetic imaging data or acousto-electromagnetic imaging data.

24. A system for ultrasound mediated electric or magnetic signal acquisition from a brain of a subject, the system comprising:
a shielding structure configured to surround a portion of a head of the subject;
a plurality of sensors supported by the shielding structure and configured to engage the head of the subject to acquire at least one of electric or magnetic signals originating in the brain of the subject;
at least one ultrasound transducer configured to deliver acoustic energy to the head of the subject to target neural activity in the brain, wherein the acoustic energy is configured to be spatially selective by focusing of the acoustic energy and temporally selective by adjusting an acoustic time window of the energy;
a processor configured to receive the at least one of electric and magnetic signals acquired by the plurality of sensors and demodulate the at least one of electric and magnetic signals using information about the acoustic energy to determine a signal associated with neural activity in the brain at a selected spatial location at a selected time.

25. The system of claim 24, wherein the plurality of sensors include at least one of electroencephalography (EEG) sensors or magnetoencephalography (MEG) sensors.

26. The system of claim 24, further comprising at least one of light blinds and a transparent slide to facilitate alignment of the system on the head of the subject.
